(19) United States
(12) Patent Application Publication
(10) Pub. No.: US 2009/0234461 A1
(43) Pub. Date: Sep. 17, 2009

(54) Apparatus and method for use of a biosurgical prosthetic rectus sheath

(76) Inventor: Robert D. Rehnke, St. Petersburg, FL (US)

Correspondence Address: Charles Raymond Sidewell, 2032 Dolphin Blvd., South, St. Petersburg, FL, 33707, US

(21) Appl. No.: 12/075,764

(22) Filed: Mar. 14, 2008

(51) Int. Cl. A61F 2/04; A61B 1/313; A61B 17/068

(52) U.S. Cl. 623/23.65; 600/183; 227/175.1

(57) ABSTRACT

A prosthetic mesh and method of use in surgical methods for closure of abdominal incisions and prosthetics for repair of ventral hernias. The mesh provides for a new prosthetic that can be used for closure of laparotomy incisions, in patients at high risk for hernia formation, and in the repair of existing ventral hernias. The mesh utilizes existing multi laminar technology for intra peritoneal mesh, and fashions it in the shape of an intact Rectus Sheath. The mesh matrix can be made of any of the various absorbable or permanent polymer filaments that are woven or knitted into a scaffold for added strength to the abdominal wall closure during the healing phase, and for delivery of bio active substances that contribute to a biochemical milieu that contributes to favorable healing. A permanent non absorbable prosthesis will be desirable in certain circumstances and for particular patients, a slowly dissolving mesh would be ideal in most cases. The absorption of the prosthesis must be slow, approximately one to two years, thus giving enough time to preserve support for the healing abdominal wall repair, as this is the period of time over which most incisional hernias present.

### CROSS REFERENCE TO RELATED APPLICATIONS

This application claims the benefit of provisional application No. 60/906,301, filed Mar. 12, 2007.

### BACKGROUND OF THE INVENTION

1. Field of the Invention

The present invention pertains generally to a surgical prosthetic device and method of use for reinforcing and reconstructing an intact abdominal wall after its division through laparotomy. More particularly, the invention constitutes a slowly absorbable or non absorbable MESH matrix which acts as a load bearing scaffolding that contains bio-active substances that promote a milieu favorable for healing. This mesh is generally in the shape of, and matches the dimensions and functions of the Rectus Sheath.

2. Description of the Prior Art

The most common general surgical procedure performed annually is the laparotomy. Access to the peritoneal cavity through a midline incision and division of the linea alba provides access to the abdominal cavity and its contents. For operations on the stomach, duodenum, and other upper abdominal organs an epigastric incision from xiphoid to umbilicus is used; incisions from umbilicus to pubic symphasis are used for operations performed on pelvic organs such as the sigmoid colon, bladder and uterus. Operations requiring greater exposure, such as hepatic or pancreatic procedures, use midline incisions encompassing the entire length from xiphoid to pubis. Midline abdominal incisions have become the gold standard for access to the abdominal cavity in spite of one significant drawback—post operative incisional hernias. Most studies cite a 10 to 15 percent incidence of hernia formation following laparotomy. This alarming statistic begs the question, why?

What factors lead to ventral, incisional hernias? Certainly surgeons worry about poor technique in closing the abdominal wall, as much has been written on this subject. It seems that a running closure with a large gauge, non absorbable suture that avoids uneven and excessive tension (that could strangulate tissue) is indicated. Complications of healing must be avoided if low hernia rates are expected. Such problems as hematoma, seroma and infection are direct contributors to poor healing of the midline. Post operative ileus or adhesions leading to periodic partial small bowel obstructions cause increased intra abdominal pressure that tests the integrity of the midline repair. Other, indirect causes include: sepsis, hypo proteinemia, and post op pulmonary failure. Certain chronic conditions, such as diabetes, smoking, obesity, and COPD, are felt to lead to increased risk of herniation.

Unbalanced tension in the abdominal wall after closure of the midline is a factor not quite as obvious as those previously mentioned. The muscular anatomy of the abdominal wall creates force vectors that pull the midline apart during movement. The only force holding the insertion of the obliques and the rectus abdominus together is the linea alba, or fascial midline of the anterior abdomen. Once this has been divided by laparotomy incision the forces that balance the muscular tensions of the abdomen will never be the same. There will always be an inherent weakness to the abdominal wall at the midline. Closure at the end of the procedure approximates the left and right sides of the rectus muscle and brings the fascial edges of the linea alba together. However, any imperfection of healing of the seam can lead to a pressure leak that can progress to a fascial defect. Over time the unbalanced pressures of the abdominal wall force vectors act to erode the closure of the abdomen at its weakest spot—the repaired linea alba. In this fashion hernia defects grow over time, explaining why the incidence of hernia diagnosis is greater at ten years post op than that seen in the first two years.

The final factor in hernia formation is poor tissue quality. Patients who develop hernias after laparotomy tend to develop recurrent hernias after hernia repairs; this in large part can be attributed to poor quality tissue. The variation in tensile strength of fascia and the relative thickness of fascia present for suturing is observed clinically. Even when strong sutures are placed correctly, weak tissue allows migration of the suture through the soft tissue which leads to loosening of the closure, separation of the midline, and hernia formation. All other factors being the same, patients with strong tissue will hold the placement of the suture until healing of the midline has occurred and avoid hernia formation.

Over ten percent of all laparotomies will develop ventral hernias leading to over 350,000 ventral hernia repairs per year in the U.S. alone. Despite advances in the art of hernia repair, over the last century, there still is a need for improvement. Historically ventral hernias were treated with primary closure, a practice that achieved no greater than a 50% success rate. Large hernias were closed with a variety of cadaveric or autologous tissue grafts or flaps without much better success. Finally techniques using synthetic mesh, as an inlay or on lay, lead to improved outcomes. Unfortunately covering fascial defects with mesh often led to complications such as infection, entero-cutaneous fistulas, chronic pain, recurrent partial small bowel obstructions, and still unacceptably high recurrence rates approaching 25%.

French surgeons Rives' and Stopa developed an approach in the early 1970's, which addressed these concerns and is still widely used in Europe today. The “French” procedure as it came to be known consists of placement of a non-absorbable mesh over closure of the posterior rectus sheath. Once the hernia defect is debrided of scar tissue, the posterior rectus sheath is separated from the anterior rectus sheath and muscle, and a space is dissected behind the rectus muscles. Once the posterior rectus sheath is pulled together and sutured, a mesh is cut to size and pulled into place by stay sutures which are passed behind the muscle and anchored to the three layers of the abdominal wall—lateral to Spigal's line. The rectus muscles and anterior rectus sheath is then closed at the midline over the mesh layer. The key to this procedure is a re-enforcing layer of mesh within a mid line closure of the defect. It avoids exposure of the bowel to mesh and restores the abdominal muscles to their natural form and function. It is limited in its effectiveness by the fact that it closes the midline under tension (relying on the strength of the mesh layer to overcome this weakness). This breaks a cardinal rule of hernia repair and requires bridge gap placement of absorbable mesh when the posterior and anterior rectus fascial layers can not be safely brought together. This, in effect, creates a controlled diastasis and limits the success of the repair. As a result, the French repair has had limited popularity in the U.S.

Modern advances in the treatment of ventral hernia were realized towards the end of the 20^{th} century when two novel techniques were developed: Laparoscopic Ventral Hernia Repair (LVHR) and Components Separation Technique. The former evolved from the expansion of the minimally invasive laparoscopic movement in General Surgery. It was aided by a new advancement in prosthetic materials—dual plane mesh. It is performed through a “closed” laparoscopic technique utilizing the placement of an overlapping mesh inlay patch of the fascial defect—a so called “no tension” repair. The mesh, which has a smooth inner layer to prevent adhesions to bowel and a textured outer layer to anchor to the abdominal wall, is designed to overlap the edge of the defect by three to five centimeters. The Components separation Technique (CST) was developed by the noted plastic surgeon Oscar Ramirez in 1990. It utilizes a midline approach with extensive undermining to raise skin flaps that expose the anterior abdominal wall. A long relaxing incision just lateral to Spegil's line separates the external oblique (EO) from the inner three layers of the abdominal wall (internal oblique (IO), transversus abdominus, and transversalis fascia). Blunt dissection in the fascial cleft between EO and IO layers allows the mobilization of composite myofascial flaps towards the midline for primary closure without undue tension. This procedure closes the defect with dynamic living tissue that restores abdominal function and aesthetics. Both techniques in experienced hands have been demonstrated in the surgical literature to be safe and effective (recurrence rates approaching 5%).

Both Laparoscopic and CST, repairs have their advantages and draw backs. LVHR is noninvasive and shown to have shortened hospital stays. It has decreased morbidity and mortality when compared to open ventral hernia techniques. However, it requires placement of expensive and potentially dangerous prosthetic mesh inside the peritoneal cavity. Additionally, it does not truly repair the abdominal wall; but instead patches the defect. Without closure of the rectus muscles to their midline approximation there can not be restoration of abdominal function and aesthetics. CST restores form and function without the need for intra-peritoneal mesh, but is an invasive procedure, which is time consuming and difficult to perform, with high rates of wound complications. This explains why the clear majority of hernia repairs are still performed with some form of prosthetic mesh repair.

Advances in hernia repair since the turn of the century have focused on improving the technology of mesh design. The development of modern mesh materials has introduced the concept of lightweight mesh and bi laminar mesh designs. These developments address some of the problems experienced with mesh over the last fifty years. In addition to recurrence of the hernia, mesh repairs can be complicated by stiffening and contracture of the prosthetic device which leads to discomfort and reduced flexibility of the abdominal wall. Additionally, intra peritoneal positioning of prosthetic can lead to intestinal adhesion, fistualization and infection. These modern prosthetics have a lightweight, flexible abdominal wall layer and a resorbable biomaterial inner layer to prevent adhesions. Two examples of modern mesh prosthetics are Ethicon's Proceed, which is a lightweight polypropylene mesh with an oxidized regenerated cellulose inner layer, and Sofradim's Parietex Composite mesh, which is woven polyester with an inner collagen based layer to prevent adhesions. This new generation of prosthetics holds the promise of safe intra peritoneal mesh placement. These modern meshes have inspired a rich and crowded intellectual property field, of which the following is an example.

Lichtenstein, et al., U.S. Pat. No. 5,593,441, January 1997, taught the use of a composite or laminated prosthesis for the repair of ventral abdominal wall defects. They described a mesh fabric layer, such as polypropylene, that would allow in growth of tissue to align with the outer abdominal wall layer and an inner layer of silastic that acts as a physical barrier to adhesion formation with abdominal viscera. The prosthetic mesh is custom cut and sized at the time of surgery to fit the defect in the abdominal wall.

Ethicon markets a tissue separating mesh for open and laparoscopic incisional hernia repair, PROCEED. It is made of a thin absorbable layer of oxidized regenerated cellulose fabric to separate abdominal viscera from the strong supportive non absorbable mesh made of soft polypropylene. The two are bonded together with absorbable polydioxanone.

Pendharkar, et al., U.S. patent application Ser. No. 401,030, April 2006, describes a process by Johnson and Johnson, Ethicon for making multi-layered fabric comprising an absorbable woven inner layer such as oxidized regenerated cellulose attached to an absorbable reinforced non woven fabric made of aliphatic polyester polymers. Additionally the non woven layer can contain pharmacologic and biologically active agents such as antibiotics and antimicrobial agents, wound healing agents, growth factors, analgesics, and acts as a scaffold for cell cultures.

Janis, et al., describes the use of an implantable mesh coupled with an anti-inflammatory such as a NSAID to prevent visceral adhesions, in the Nov. 9, 2006 patent application #20060251702. They also call for the prosthetic device to include absorbable extra cellular matrix materials (ECMs) such as submucosa and other natural and fully synthetic growth factors.

Michael Milbocker has described a new device in patent application #20060233852, Oct. 19, 2006, which calls for a prosthetic sheet made of a non absorbable hydrogel reinforced with fiber, such that the fiber is encapsulated and protected from interaction with tissue. The sheet is laser punched to allow a desirable amount of tissue through-growth.

These multi-layered laminated composite prostheses address the make up of the prosthetic sheets, but fail to understand the design shapes and dimensions needed for the paradigm shift from patch repair to reconstruction of an intact abdominal wall.

What are needed are improved approaches to closure of the abdomen, which take into account the high risk of hernia formation, and an improved approach to repair of ventral hernias once they have formed. The object of the current invention addresses the need for a new prosthetic mesh designed for this enlightened approach. It realizes the factors responsible for hernia formation and addresses these with the full armamentarium of modern technologies which are included in a novel dimensional design tailored to its use.

Accordingly, there is a need for improved surgical method and apparatus for use in surgery for minimally invasive surgical ventral hernia repair to overcome the aforementioned disadvantages in the prior art.

The use of surgical methods and mesh fabrics of known designs and configurations is known in the prior art. More specifically, known designs and configurations heretofore devised and utilized are known to consist basically of familiar, expected, and obvious structural configurations and methods, notwithstanding the myriad of designs encompassed by the crowded prior art which has been developed for the fulfillment of countless objectives and requirements.

While these devices fulfill their respective, particular objectives and requirements, the aforementioned patents do not describe a method of Laparoscopic Ventral Hernia Repair (LVH Repair) that is an improvement on its predecessor the laparoscopic patch inlay through Components Separation Technique (CST) using minimally invasive techniques and a surgical apparatus for use in reconstruction of an intact Biosurgical Prosthetic Rectus Sheath.

Therefore, it can be appreciated that there exists a continuing need for a new and improved surgical method and apparatus for use in ventral hernia repair in a minimally invasive manner. In this regard, the present invention substantially fulfills this need.

In this respect, the apparatus and method for use of a biosurgical prosthetic rectus sheath according to the present invention substantially departs from the conventional concepts and designs of the prior art, and in doing so provides an apparatus and primarily developed for the purpose of reinforcing and reconstructing an intact abdominal wall after its division through laparotomy comprising a mesh is generally in the shape of, and matches the dimensions and functions of the Rectus Sheath

Therefore, it can be appreciated that there exists a continuing need for a new and improved apparatus and method for use of a biosurgical prosthetic rectus sheath wherein the matches the dimensions and functions of the Rectus Sheath. In this regard, the present invention substantially fulfills this need.

### SUMMARY OF THE INVENTION

In view of the foregoing disadvantages inherent in the known surgical methods for closure of abdominal incisions and prosthetics for repair of ventral hernias, the present invention provides for a new prosthetic mesh. It can be used routinely for closure of laparotomy incisions, in patients at high risk for hernia formation, and in the repair of existing ventral hernias.

To attain this, the present invention takes advantage of the existing multi laminar technology for intra peritoneal mesh, and fashions it in the shape of an intact Rectus Sheath. The mesh matrix can be made of any of the various absorbable or permanent polymer filaments that are woven or knitted into a scaffold for added strength to the abdominal wall closure during the healing phase, and for delivery of bio active substances that contribute to a biochemical milieu that contributes to favorable healing.

A permanent non absorbable prosthesis will be desirable in certain circumstances and for particular patients, a slowly dissolving mesh would be ideal in most cases. The absorption of the prosthesis must be slow, approximately one to two years, thus giving enough time to preserve support for the healing abdominal wall repair, as this is the period of time over which most incisional hernias present.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved sex aid which has all the advantages of the prior art a surgical prosthetic device and method of use for reinforcing and reconstructing an intact abdominal wall after its division through laparotomy wherein the invention constitutes a slowly absorbable or non absorbable MESH matrix which acts as a load bearing scaffolding that contains bio-active substances that promote a milieu favorable for healing such that the mesh is generally in the shape of, and matches the dimensions and functions of the Rectus Sheath of known designs and configurations and none of the disadvantages.

It is another object of the present invention to provide a new and improved a surgical prosthetic device and method of use for reinforcing and reconstructing an intact abdominal wall after its division through laparotomy wherein the invention constitutes a slowly absorbable or non absorbable MESH matrix which acts as a load bearing scaffolding that contains bio-active substances that promote a milieu favorable for healing such that the mesh is generally in the shape of, and matches the dimensions and functions of the Rectus Sheath which may be easily and efficiently manufactured and marketed.

It is a further object of the present invention to provide a new and improved a surgical prosthetic device and method of use for reinforcing and reconstructing an intact abdominal wall after its division through laparotomy wherein the invention constitutes a slowly absorbable or non absorbable MESH matrix which acts as a load bearing scaffolding that contains bio-active substances that promote a milieu favorable for healing such that the mesh is generally in the shape of, and matches the dimensions and functions of the Rectus Sheath. which is of a durable and reliable construction.

An even further object of the present invention is to provide a new and improved a surgical prosthetic device and method of use for reinforcing and reconstructing an intact abdominal wall after its division through laparotomy wherein the invention constitutes a slowly absorbable or non absorbable MESH matrix which acts as a load bearing scaffolding that contains bio-active substances that promote a milieu favorable for healing such that the mesh is generally in the shape of, and matches the dimensions and functions of the Rectus Sheath. which is susceptible of a low cost of manufacture with regard to both materials and labor.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings.

FIG. 1 is perspective view of the Biosurgical Prosthetic Rectus Sheath constructed in accordance with the principles of the present invention.

FIG. 2 is a cross sectional view of intra peritoneal placement and anchorage of the present invention to the four layers of the abdominal wall.

FIG. 3 is an illustration of a midline hernia defect.

FIG. 4 is a minimally invasive balloon dissector in use according to the present invention.

FIG. 5 illustrates a balloon dissection between External Oblique and Internal Oblique muscles according to the present invention.

FIG. 6a illustrates an alternate mechanical blunt dissection of external from internal oblique muscle according to the present invention.

FIG. 6b is an alternative view of the mechanical blunt dissection of external from internal oblique muscle according to the present invention show in FIG. 6a.

FIG. 7 Illustrates an endoscopic release of the abdominal components according to the present invention.

FIG. 8 illustrates a laparoscopic approximation of the midline rectus fascia according to the present invention.

FIG. 9 illustrates a laparoscopic closure of the midline with stapling device according to the present invention.

FIG. 10 illustrates a reconstructed rectus sheath after endoscopic placement and anchorage according to the present invention.

FIG. 11 illustrates a laparoscopic anchorage of prosthetic mesh to four layers of the lateral abdominal wall according to the present invention.

The same reference numerals refer to the same parts throughout the various Figures.

### DESCRIPTION OF THE PREFERRED EMBODIMENT

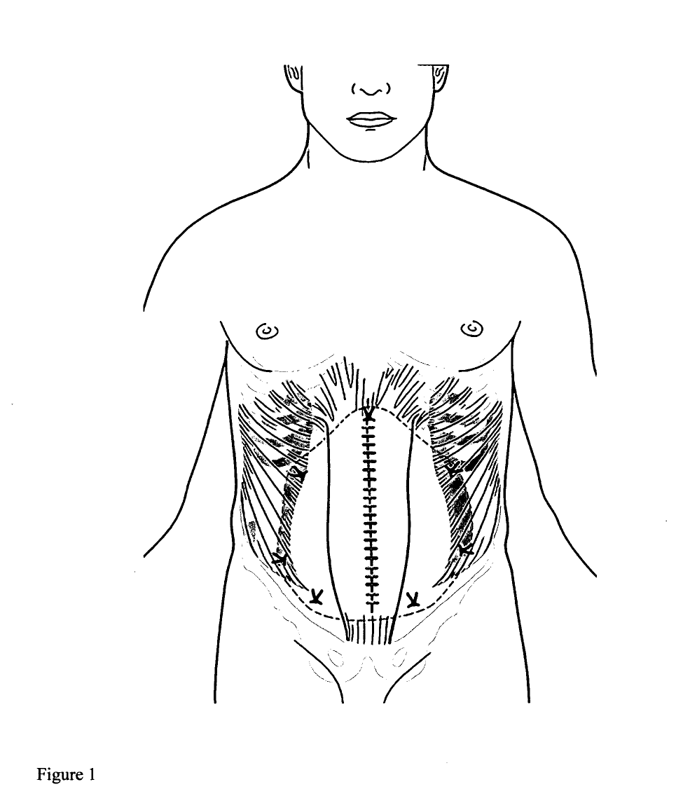

With reference now to the drawings, and in particular to FIG. 1 thereof, the preferred embodiment of the new and improved surgical prosthetic device and method of use embodying the principles and concepts of the present invention and generally designated by the reference numeral 10 will be described.

The present invention, the new and improved surgical prosthetic device and method of use, is a device 10 comprised of a plurality of components. Such components, in their broadest context, includes a prosthetic mesh 10 for use in the known surgical methods for closure of abdominal incisions and prosthetics for repair of ventral hernias. The present invention provides for a new prosthetic mesh. It can be used routinely for closure of laparotomy incisions, in patients at high risk for hernia formation, and in the repair of existing ventral hernias. Each of the individual components is specifically configured and correlated one with respect to the other so as to attain the desired objectives.

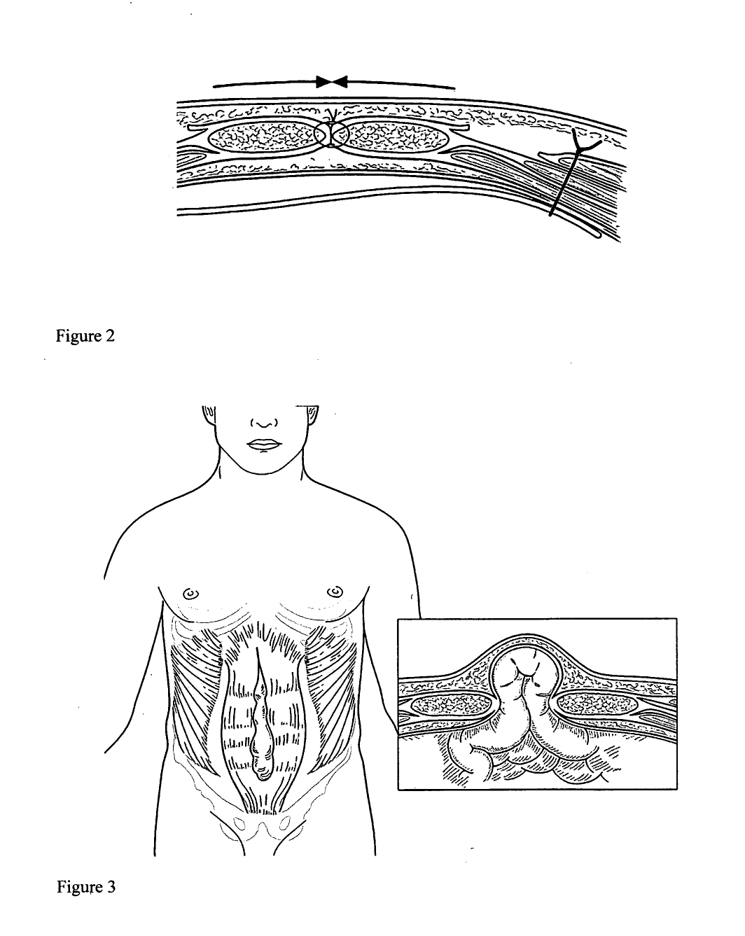

FIG. 1 shows placement of a prosthetic rectus sheath behind a midline laparotomy closure. At the completion of procedures at high risk of hernia formation, the new device 10 would be positioned in the intra peritoneal position and sutured to the four layers of the abdominal wall just lateral to the semi-lunar line (FIG. 2). These anchoring sutures 12 preferably are slowly absorbable, as is the prosthetic device, in this prophylactic use of the mesh. They are placed through small skin incisions 14 and knots 16 are buried subcutaneously. The midline rectus fascia would then be closed with suture that incorporates the mesh 10 as part of the repair.

The present invention takes advantage of the existing multi laminar technology for intra peritoneal mesh, and fashions it in the shape of an intact Rectus Sheath 18. The mesh matrix 20 can be made of any of the various absorbable or permanent polymer filaments 22 that are woven or knitted into a scaffold 24 for added strength to the abdominal wall closure during the healing phase, and for delivery of bio active substances 26 that contribute to a biochemical milieu that contributes to favorable healing.

An embodiment of the invention comprising a permanent non absorbable prosthesis will be desirable in certain circumstances. Further, a slowly dissolving mesh would be ideal in most cases. The absorption of the prosthesis must be slow, approximately one to two years, thus giving enough time to preserve support for the healing abdominal wall repair, as this is the period of time over which most incisional hernias present.

More specifically, the present invention calls for a general shape and dimension that matches the existing Rectus Sheath. This is generally the shape of a shield that runs from the sub costal region 30 to the inguinal region 32, and from left 34 to right 36 semi-lunar line. The actual Rectus Sheath is three dimensional and is made of an anterior sheath 40 that covers the superficial aspect 42 of the rectus muscles and a posterior sheath 44 that covers the deep aspect of these muscles. The posterior sheath 48 mirrors the anterior one with the exception that there is no posterior sheath below the umbilicus, or arcuate line 52.

The most important aspect of the Rectus Sheath is its fusion with the fascia of the oblique muscles at the semi-lunar line. This embodiment of the invention calls for a prosthetic multi-laminar two dimensional mesh sheet that can be placed into the peritoneal cavity. This embodiment of the invention is designed with a shape and dimension sufficient to run from costal margin to inguinal region and from semi-lunar line to semi-lunar line.

The embodiment is designed for use behind a closed or reconstructed midline linea alba and is anchored to the lateral abdominal wall with slowly absorbing or permanent sutures through all four layers of the abdomen, thus allowing for direct fusion with the fascia of the lateral obliques. This aspect is not possible with the present art. Current techniques call for use of suture closure alone of the midline linea alba. If a focal area of the closure has poor quality or missing fascia the balance of opposing oblique force vectors is lost and the midline separates.

The present invention provides for a modern mesh with anatomic dimensions to reach from lateral oblique to lateral oblique—thus assuring solid anchorage into the obliques.

The present invention provides for a prosthetic device that supports multiple bio active substances that enhance the healing process following laparotomy. This would include, but not be limited to: anti inflammatory and other anti adhesion agents to prevent adhesions of the bowel to the mesh matrix and intra peritoneal intestinal adhesions; antibiotics that would have time released activity and deliver a concentration of prophylactic antibiotics in the peritoneal fluid for the early phase of healing; growth factors such as, platelet derived growth factors, that would enhance the amount and speed of new collagen deposition and healing in the wound; coagulants, such as thrombin, that would leach into the peritoneal fluid and thus bathe the raw surfaces created during surgical procedures and prevent bleeding; and anti cancer agents such as chemotherapy drugs or radiation seeds used to treat peritoneal seeding of metastatic colon or ovarian cancers. In addition to bio active substances, time released local anesthetics can be supported by the mesh matrix and allow for prolonged pain relief. The present invention could include any or all of these bio active substances depending on the specific application or clinical need.

The present invention provides for a surgical prosthetic device which is designed to replace an intact Rectus Sheath. The importance of an intact sheath has been described but will now be further explained. The Rectus Sheath unifies and balances all the forces of the abdominal wall muscles—the rectus and lateral oblique muscles. Once the linea alba is divided surgically, the intra peritoneal forces are no longer evenly shared throughout the abdominal wall. The force vectors of lateral tension created by the oblique musculature are unopposed by the force vectors of the rectus muscles. The rectus muscles oppose the oblique force vectors only as a unified whole, which is accomplished by the integrity of the rectus sheath. Its fascial make up unifies the rectus abdominus muscles and transmits the opposing force vectors of the left oblique muscle group against the right. This balance of forces is destroyed by midline laparotomy. The existing use of surgical devices such as suture or mesh placed beyond the midline defect or repair by one to several centimeters, fails to distribute tension vectors that adequately oppose and thus balance the lateral pull of the oblique muscles. The prior art for abdominal midline closure patches a defective rectus unit. The integrity of the closure is undone by the pulling forces of the obliques and by the outward Pascal's forces of intra abdominal pressure; once the repaired midline seam is disrupted by the first, the second creates the ever expanding hernia defect. This is made evident by the fact that unbalanced tension over time leads to a higher and higher percentage of hernia formation and/or recurrence. The failure to evenly distribute abdominal wall tension is primarily due to failure in dimensional design and use of the prior art. The present invention comprises a large prosthetic mesh that is in the shape and dimension of an intact rectus sheath. The present invention allows for the intra peritoneal placement of said mesh and suture anchorage to the four layers of the lateral abdominal wall (external oblique, internal oblique, transversus abdominus, and transversalis fascia). In this way the compromised strength of a rectus unit that has been divide at the linea alba is bypassed. The newly reconstructed rectus layer is a solid unit without a seam and inserts, due to the newly designed dimensions and proscribed method of use, into the oblique muscles directly. This offloads the tension of the oblique muscles from the subsequently repaired linea alba. The new mesh invention is designed to be flexible and lightweight with a bursting strength just beyond that of an un operated abdominal wall.

Having accounted for the causative forces of hernia formation and addressed them with the present device, it can now be applied in laparotomy closures at high risk for hernia development, such as open gastric bypass procedures, and in improved techniques for incisional hernia repair. The illustrations that follow and description of the preferred use of this new device will illustrate this improved approach to closure of the abdomen and reconstruction of the abdominal wall following laparotomy.

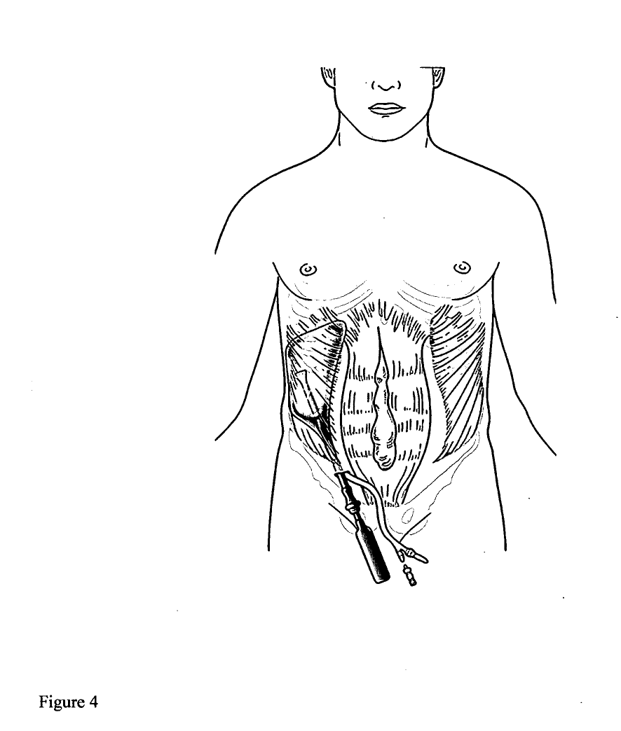

FIG. 3 represents the case of an existing incisional hernia. In the preferred embodiment the midline is reconstructed thus restoring form and function to the abdominal wall and the Biosurgical Prosthetic Rectus Sheath is placed intra peritoneal and anchored to the lateral abdominal wall to re enforce the repair and balance abdominal wall tensions. But first a fascial release must be performed in order to bring the rectus muscle and linea alba together without undue tension. This is accomplished through a Components Separation Technique using a minimally invasive approach (MICST). This release is required due to the lateral retraction vectors which come into play in the development of incisional hernias.

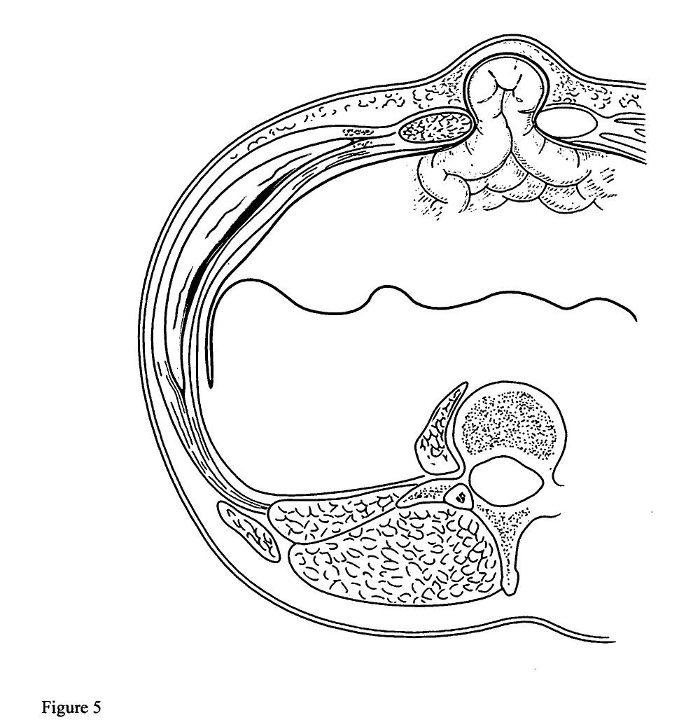

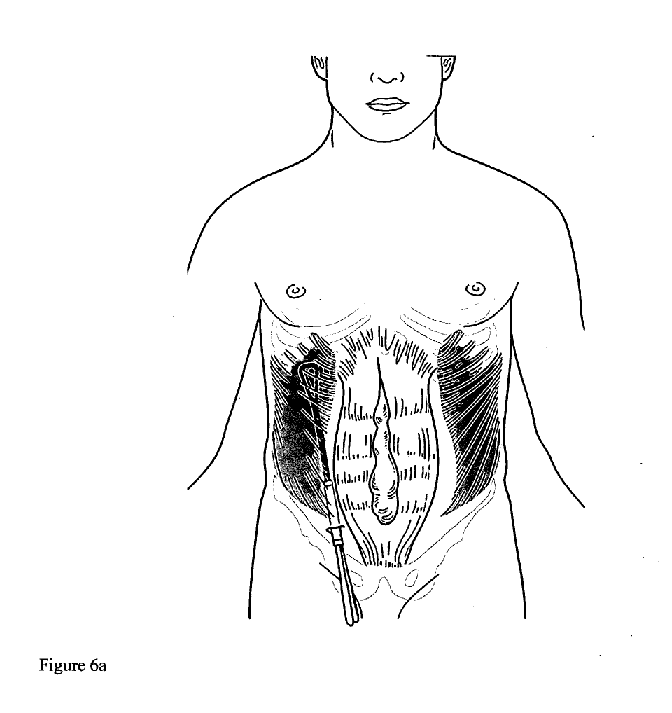

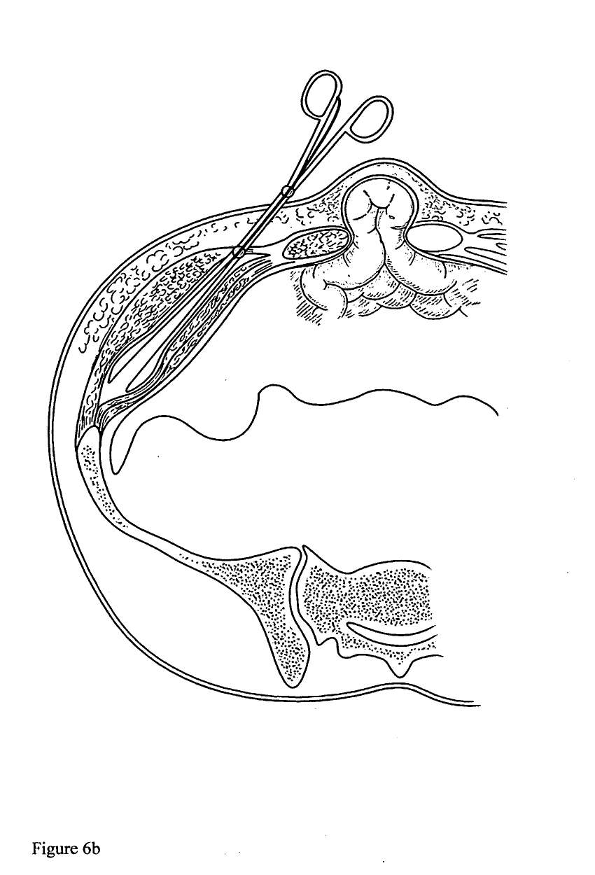

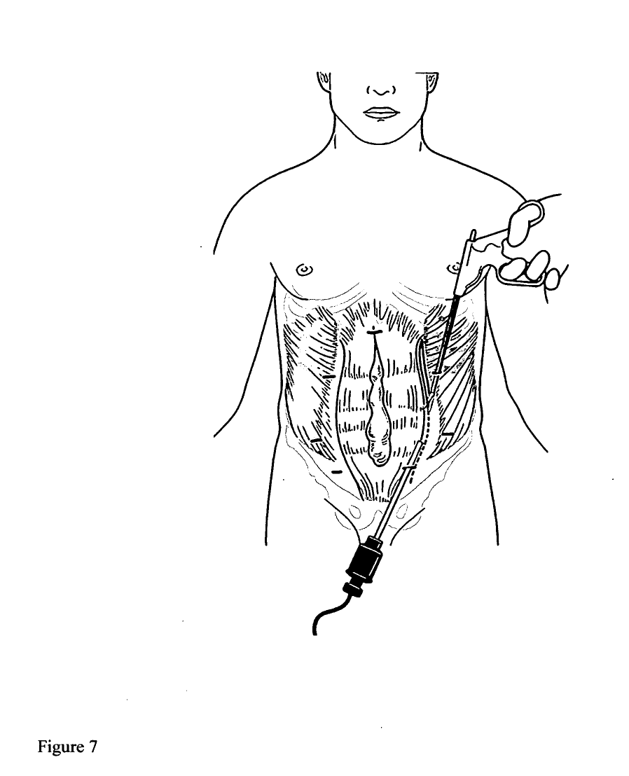

MICST is begun with a small horizontal incision just above the external inguinal ring. This anatomic region is completely familiar to all general surgeons (the same incision is used for open inguinal hernia repairs) and consistent in all body types despite the size of the ventral hernia. A small vertical incision through the deep fascia is then made just above and lateral to this point. This opens the fascial cleft between the External Oblique (EO) and Internal Oblique (IO). The surgeon's index finger performs the initial blunt dissection in this plane and verifies correct position by the ease of separation of the plane laterally but the inability to bluntly dissect medially past Spigal's line (the line of fusion of the EO and IO fascia at the lateral border of the rectus muscle—the so called semi-lunar line). Next a MICST balloon dissector is inserted into this fascial cleft and inflated until the borders of the space are reached (Spigal's line medially, above the costal margin and below the infra-mammary fold superiorly, lumbar region laterally and inguinal region inferiorly)—FIGS. 4 and 5. Alternatively a mechanical blunt dissector can be used to perform the minimally invasive components separation instead of balloon dissectors (FIGS. 6a and 6b). This step is then repeated on the contra-lateral side of the abdomen. Next, the balloon dissectors are removed and a 15 mm. laparoscopic trocar is inserted through the inguinal incision. It is used to insufflate CO2 gas and inflate the endoscopic space. A 10 mm. laparoscope is then inserted into the fascial cleft between EO and IO. Under endoscopic visualization, 10 mm. trocars are inserted at the four corners of the anterior abdominal wall, approximately four centimeters lateral to the semi-lunar line. Endoscopic electro-cautery scissors are used to divide the EO fascia two centimeters lateral to the semi-lunar line. The superior release of this fascia is performed by inserting the endoscopic scissors through the inferior “four corners” incision, while the inferior fascial release is performed through the superior “four corners” trocar (FIG. 7). These steps are then repeated on the contra-lateral side. This is a safe, a-vascular region so this maneuver is quick and easy to perform. These maneuvers constitute a Minimally Invasive Components Separation.

Part B of the LVH Repair involves the laparoscopic closure of the midline using new laparoscopic fascial graspers and a new laparoscopic rectus fascial stapler. Finally, the Biosurgical Rectus Sheath is stapled in place along the posterior side of the abdominal wall, and its lateral and superior edges are tied to the abdominal wall at the laparoscopic access ports.

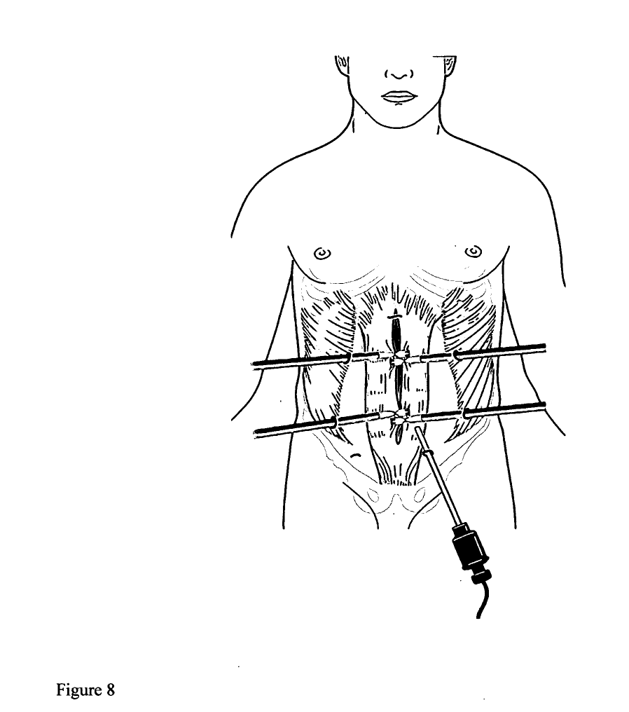

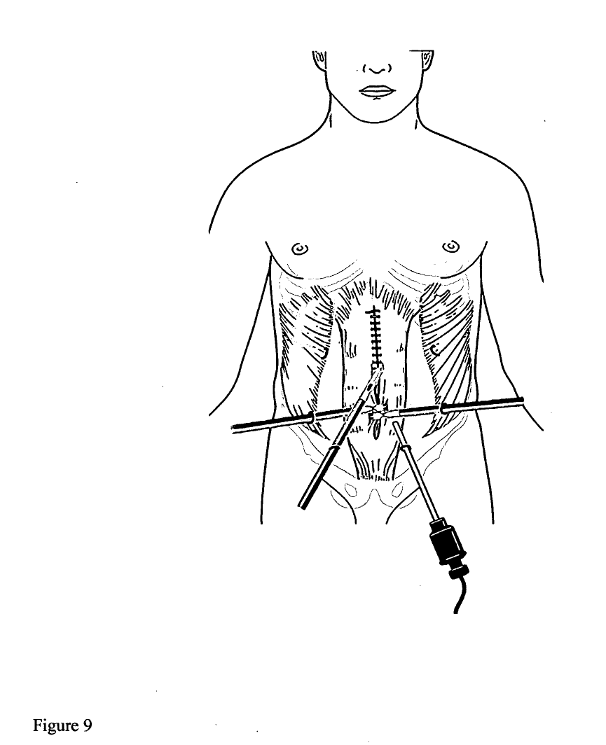

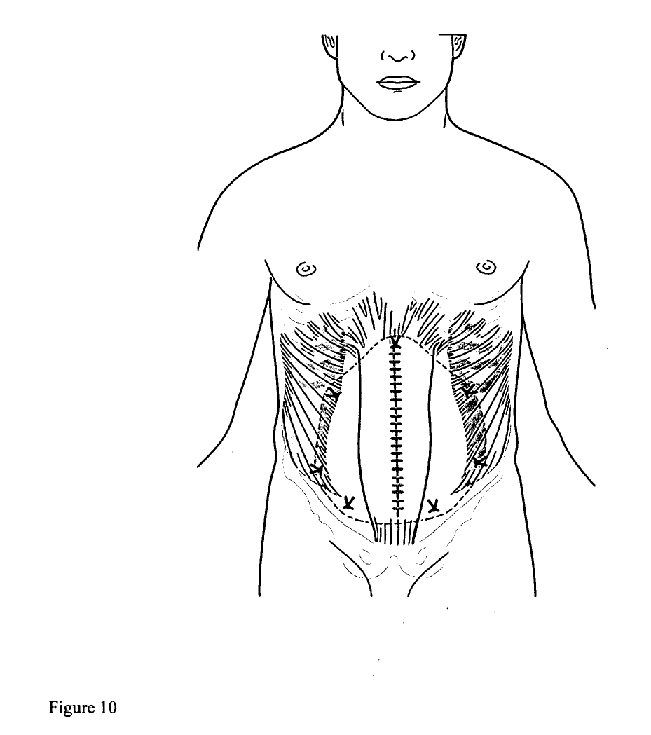

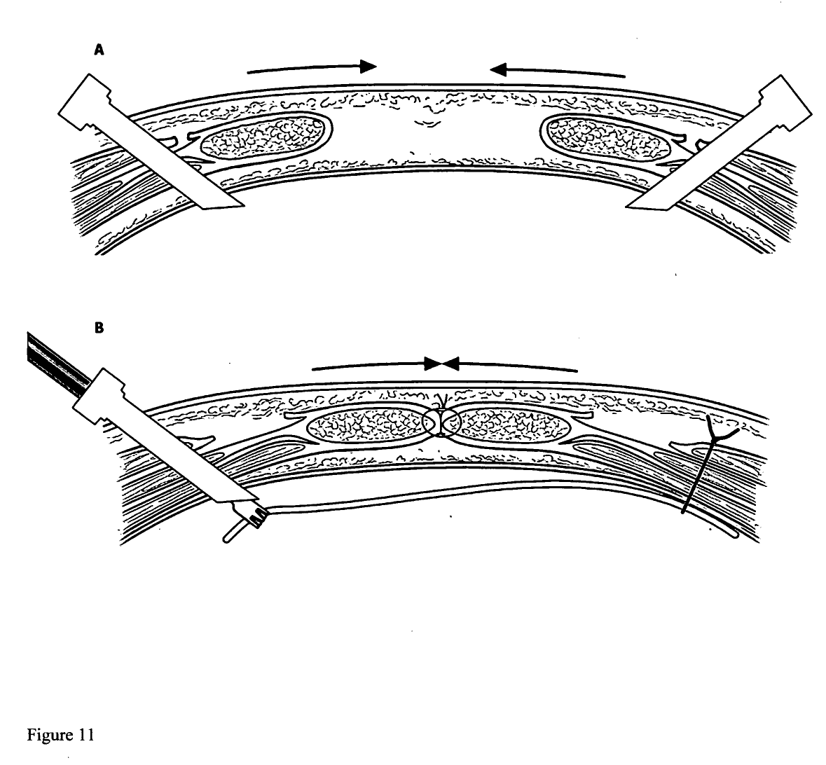

This is accomplished by inserting the 15 mm. trocars through the remaining layers of the abdominal wall in a more medial and superior direction. The peritoneal cavity is then insufflated with CO2 gas. A third 15 mm. trocar is placed in the epigastric midline to establish a triangulation of large ports. The 10 mm. trocar ports, at the four corners of the anterior abdominal wall, are then inserted through the remaining layers into the peritoneal cavity. A 10 mm laparoscope is inserted through one of the inguinal ports and a Rectus Fascial Stapler through the other. Fascial graspers are inserted into all four corners and placed across the midline in order to pull the contra-lateral midline to the center (FIG. 8). The Rectus Fascial Stapler is then used to staple the midline fascia together in the upper half of the defect. The Rectus Fascial Stapler is then switched to the epigastric port and the lower half of the defect is stapled together (FIG. 9). Next, the Biosurgical Rectus Sheath is introduced into the peritoneal cavity and unfurled with the anti adhesion side towards the viscera. It is centered over the midline and stapled to the posterior rectus sheath. The lateral wings of the mesh are then anchored to the full thickness of the lateral abdominal wall through the “four corners” trocar sites, after they are pulled back and re inserted through the advanced abdominal layers (FIG. 10). These sutures are tied through the lateral edge of the released External Oblique fascia for maximal strength (FIG. 11). In this way the tension of the intra abdominal cavity is transmitted to the lateral components and offloads tension from the midline repair. Closure of the fascia at the large ports also incorporates the mesh to insure anchoring of the mesh to the abdominal wall superiorly and inferiorly. The lateral anchoring of the mesh is designed to be closed under tension so that it will be taught when insulflation is released. Reinforcement of the midline is provided through the mesh inlay's baffling nature, and accomplishes the ultimate in achieving the principle of “overlapping” the defect. It also protects against the potential weakness in the region of the components separation relaxing incision. This is done by covering this area with the lateral wings of the mesh (from the intra abdominal side) and anchoring it to all four layers of the abdominal wall. Finally, three anchoring sutures are placed along the old midline scar as a mass closure of the linea alba (including the mesh layer).

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A new and improved surgical prosthetic device and method of use for reinforcing and reconstructing an intact abdominal wall comprising, in combination:
A prosthetic mesh for use in the known surgical methods for closure of abdominal incisions and prosthetics for repair of ventral hernias wherein the mesh has a shape and dimension that matches a human rectus sheath.

2. The new and improved surgical prosthetic device and method of use for reinforcing and reconstructing an intact abdominal wall as set forth in claim 1 wherein the mesh comprises an anterior sheath for covering a superficial aspect of the rectus muscles and a posterior sheath for covering a deep aspect of the rectus sheath such that the posterior sheath mirrors the anterior sheath with the exception that there is no posterior sheath below an umbilicus line thereof.

3. The new and improved surgical prosthetic device and method of use for reinforcing and reconstructing an intact abdominal wall as set forth in claim 1 wherein the mesh with anatomic dimensions reaches from lateral oblique to lateral oblique—thus assuring solid anchorage into the obliques.

4. The new and improved surgical prosthetic device and method of use for reinforcing and reconstructing an intact abdominal wall as set forth in claim 1 wherein The mesh supports multiple bio active substances that enhance the healing process following laparotomy.

5. A new and improved surgical prosthetic device and method of use for reinforcing and reconstructing an intact abdominal wall comprising an improved approach to closure of the abdomen and reconstruction of the abdominal wall following laparotomy comprising the following steps for cases with existing incisional hernia, in combination:
reconstructing the midline thereby restoring form and function to the abdominal wall by positioning a prosthetic rectus sheath intra peritoneal;
performing a fascial release to bring the rectus muscle and linea alba together without undue tension through a Components Separation Technique using a minimally invasive approach (MICST);
anchoring to the prosthetic rectus sheath to a lateral abdominal wall to reinforce the repair and balance abdominal wall tensions; and
Stapling the Biosurgical Rectus Sheath in place along the posterior side of the abdominal wall, and its lateral and superior edges are tied to the abdominal wall at the laparoscopic access ports.

6. The new and improved surgical prosthetic device and method of use for reinforcing and reconstructing an intact abdominal wall comprising an improved approach to closure of the abdomen and reconstruction of the abdominal wall following laparotomy as set forth in claim 5 wherein the MICST comprises the following steps:
Beginning the MICST with a small horizontal incision just above an external inguinal ring;
Making a small vertical incision through a deep fascia just above and lateral to this point;
Opening the fascial cleft between the External Oblique (EO) and Internal Oblique (IO);
Performing an initial blunt dissection with a surgeon's index finger in a plane and verifying correct position by the ease of separation of the plane laterally but the inability to bluntly dissect medially past Spigal's line;
Inserting a MICST balloon dissector is inserted into the fascial cleft and inflating until the borders of the space are reached;
Repeating the dissection on a contra-lateral side of the abdomen;
Removing the balloon dissectors are removed and inserting a 15 mm. laparoscopic trocar is inserted through the inguinal incision;
Isufflating CO2 gas and inflating the endoscopic space;
Inserting a 10 mm. laparoscope into fascial cleft between EO and IO;
Inserting trocars under endoscopic visualization at the four corners of the anterior abdominal wall, approximately four centimeters lateral to the semi-lunar line;
Using Endoscopic electro-cautery scissors are used to divide the EO fascia two centimeters lateral to the semi-lunar line;
Performing the superior release of this fascia is by inserting the endoscopic scissors through the inferior “four corners” incision, while the inferior fascial release is performed through the superior “four corners” trocar (FIG. 7)'
Repeating these steps on the contra-lateral side.

7. The new and improved surgical prosthetic device and method of use for reinforcing and reconstructing an intact abdominal wall comprising an improved approach to closure of the abdomen and reconstruction of the abdominal wall following laparotomy as set forth in claim 5 wherein the MICST comprises the following steps:
Stapling the laparoscopic closure of the midline with laparoscopic fascial graspers and laparoscopic rectus fascial stapler wherein the stapling is accomplished by inserting trocars through the remaining layers of the abdominal wall in a medial and superior direction wherein the peritoneal cavity is insufflated with CO2 gas and a third trocar is placed in the epigastric midline to establish a triangulation of large ports;
Trocar ports are then inserted through the remaining layers into the peritoneal cavity at the four corners of the anterior abdominal wall;
A 10 mm laparoscope is inserted through one of the inguinal ports and a Rectus Fascial Stapler through the other;
Fascial graspers are inserted into all four corners and placed across the midline in order to pull the contra-lateral midline to the center;
The Rectus Fascial Stapler is then used to staple the midline fascia together in the upper half of the defect;
The Rectus Fascial Stapler is then switched to the epigastric port and the lower half of the defect is stapled together;
The Biosurgical Rectus Sheath is introduced into the peritoneal cavity and unfurled with the anti adhesion side towards the viscera and is centered over the midline and stapled to the posterior rectus sheath.;
The lateral wings of the mesh are then anchored to the full thickness of the lateral abdominal wall through the “four corners” trocar sites, after they are pulled back and re inserted through the advanced abdominal layers;
Sutures are tied through the lateral edge of the released External Oblique fascia for maximal strength such that tension of the intra abdominal cavity is transmitted to the lateral components and offloads tension from the midline repair.
Finally, three anchoring sutures are placed along the old midline scar as a mass closure of the linea alba (including the mesh layer).
